(19) United States
(12) Patent Application Publication
(10) Pub. No.: US 2007/0137669 A1
(43) Pub. Date: Jun. 21, 2007

(54) Hair coloring system including an applicator tip

(76) Inventors: Carol Hamilton, New York, NY (US); Kathy Roher, Hartsdale, NY (US); Celine Brucker-Doessant, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/543,073

(22) Filed: Oct. 5, 2006

Related U.S. Application Data
(60) Provisional application No. 60/751,301, filed on Dec. 19, 2005.

(51) Int. Cl. A61K 8/18

(52) U.S. Cl. 132/208

(57) ABSTRACT

A hair coloring system includes at least one component of a hair coloring product and a container configured to contain the hair coloring product. The container may include a neck having a connecting portion and a top neck end. The system may further include an applicator tip for applying the hair coloring product to hair. The applicator tip may include a base portion configured to connect the applicator tip releasably to the connecting portion of the neck of the container and a nozzle portion extending from the base portion to a top end of the tip. The applicator tip and the container may be configured such that when the applicator tip is fully connected to the connecting portion of the neck of the container, an axial length from the top neck end to the top end of the tip is greater than about 1.9 inches. In some examples, the applicator tip and the container may be configured such that a ratio of the axial length to an outer diameter of a portion of the neck including the top neck end is greater than about 1.8.

The present application claims the benefit of priority under 35 U.S.C. § 119(e) to U.S. provisional patent application No. 60/751,301, filed Dec. 19, 2005.

### BACKGROUND OF THE INVENTION

1. Field of the Invention

The present invention generally relates to hair coloring products. In particular, some aspects of the invention relate to hair coloring systems including an applicator tip for dispensing a hair coloring product and related methods of using such systems.

2. Description of the Related Art

Hair coloring products include both hair dyes (e.g., substances that act to darken hair color) and hair lighteners (e.g., substances, such as bleaches and at least some highlighters, that act to lighten hair color). Some of these products may be applied using an applicator. Sometimes the applicator may be in the form of a relatively short dispensing tip packaged together with other parts of a hair coloring kit. These kits may be marketed for consumer (non-professional) use, and thus, may be available for sale to consumers in stores for in-home use by individuals applying a hair coloring product to their own hair.

The applicator tips provided in conventional consumer hair coloring systems generally have a relatively short tip length in order to accommodate ease of manufacture, lower cost, reduced weight, packaging space limitations, etc. However, with this short tip length it may be difficult to apply hair coloring products to certain types of hair, such as hair that is thick, curly, and/or long. In particular, it may be difficult for an individual to part their hair and/or locate the dispensing end of the applicator tip adjacent to their scalp or roots.

Further, use of short tips may result in hair coloring product transfer from the hair onto the outer surface of a base of the applicator tip and/or to the container from which the product is being dispensed. This may result in unwanted transfer of product to skin, clothing, or other items during use, and may result in generally messy conditions.

In addition, short tips may be more prone to undesired leaking and/or dripping of product from the dispensing opening. This may also result in generally messy conditions, and thus, unwanted transfer of product to skin, clothing or other items.

In view of the above-mentioned issues, at least some current kits and/or techniques may be cumbersome, inefficient, and generally time consuming. For these and other reasons, there is a need for alternative approaches that may at least partially overcome one or more of the above-outlined drawbacks and/or limitations of current arrangements.

Although the present invention may obviate one or more of the above-mentioned drawbacks and/or limitations, it should be understood that some aspects of the invention might not necessarily obviate one or more of those drawbacks and/or limitations.

### SUMMARY

In the following description, certain aspects and embodiments of the present invention will become evident. It should be understood that the invention, in its broadest sense, could be practiced without having one or more features of these aspects and embodiments. In other words, these aspects and embodiments are merely exemplary.

One aspect of the invention relates to a hair coloring system. The system may include at least one component of a hair coloring product. The system may also include a container configured to contain the hair coloring product. The container may include a neck having a connecting portion and a top neck end. The system may further include an applicator tip for applying the hair coloring product to hair. The applicator tip may include a base portion configured to connect the applicator tip releasably to the connecting portion of the neck of the container. The applicator tip may also include a nozzle portion extending from the base portion to a top end of the tip. In addition, the applicator tip and the container may be configured such that when the applicator tip is fully connected to the connecting portion of the neck of the container, an axial length from the top neck end to the top end of the tip is greater than about 1.9 inches. Further, the applicator tip and the container may be configured such that a ratio of said axial length to an outer diameter of a portion of the neck including the top neck end is greater than about 1.8.

In some examples, the axial length may be greater than about 2.3 inches and the ratio may be greater than or equal to about 2.1. In some examples, the tip may have an overall axial height greater than about 2.7 inches.

In at least some embodiments, the applicator tip may further include a seal configured to extend into the neck of the container when the applicator tip is fully connected to the connecting portion. In some examples, the seal may have an outer diameter of less than about 1.3 inches.

The applicator tip may also include an opening at the top end of the tip. In some embodiments, the applicator tip may include a tab configured to be manually removed to form the opening at the top end of the applicator tip.

An inner surface of the applicator tip may define a flow path. In some examples, the nozzle portion may include axially-oriented ribs extending along the inner surface. The nozzle portion may also include an outer surface and at least one of the inner surface and the outer surface may be tapered, in an axial direction, at an angle of about 8.7 degrees.

In at least some embodiments, the system may include one or more additional items, such as a pair of gloves, at least one additional component of the hair coloring product, and instructions for using the hair coloring system to color hair. The system may be packaged in a packaging. The components of the hair coloring product, the container, the applicator tip, and the pair of gloves may be contained in the packaging.

Another aspect relates to a method of coloring hair performed by an individual. The method may include dispensing hair coloring product from an opening at the top end of the applicator tip. The method may further include placing the opening adjacent to the individual's scalp so as to apply the hair coloring product to the individual's hair. The method may further include using the applicator tip to form a part in the individual's hair.

Yet another aspect may relate to a hair coloring system including first and second components, wherein the first and second components form a hair coloring product by mixing the first and second components together. The system may also include a container configured to contain the hair coloring product. The container may include a neck having a connecting portion and a top neck end, wherein an outer diameter of a portion of the neck including the top neck end is less than about 1.5 inches. The system may further include an applicator tip for applying the hair coloring product to hair. The applicator tip may include a base portion configured to connect the applicator tip releasably to the connecting portion of the neck of the container. The applicator tip may also include a nozzle portion extending from the base portion to a top end of the tip. The system may also include a packaging, wherein the first and second components of the hair coloring product, the container, and the applicator tip are contained in the packaging. In addition, the applicator tip and the container may be configured such that when the applicator tip is fully connected to the connecting portion of the neck of the container, an axial length from the top neck end to the top end of the tip may be greater than about 1.9 inches. In some examples, the outer diameter of the top neck end may be less than or equal to about 1.2 inches.

Aside from the structural and procedural arrangements set forth above, the invention could include a number of other arrangements such as those explained hereinafter. It is to be understood that both the foregoing description and the following description are exemplary only.

### BRIEF DESCRIPTION OF THE DRAWINGS

The accompanying drawings are incorporated in and constitute a part of this specification. The drawings illustrate exemplary embodiments and, together with the description, serve to explain some principles of the invention. In the drawings,

FIG. 1A is a partially schematic, cross-section view of an exemplary applicator tip connected to a container;

FIG. 1B is a detailed view of portion 1B of FIG. 1A;

FIG. 1C is a top view of the tip of FIG. 1A;

FIG. 1D is a side view of the tip of FIG. 1A;

FIG. 1E is a bottom view of the tip of FIG. 1A;

FIG. 2A is a cross-sectional view of an end portion of an alternative embodiment including a removable tab on an applicator tip having features of the tip of FIG. 1A;

FIG. 2B is a cross-sectional view taken along line 2B-2B of FIG. 2A;

FIG. 3 is a partially schematic, perspective view showing an exemplary embodiment of a system including the exemplary tip of FIG. 1A; and

FIGS. 4A, 4B, and 4C show exemplary uses of parts of the system of FIG. 3 to apply hair coloring to hair.

### DESCRIPTION OF EXEMPLARY EMBODIMENTS

Reference will now be made in detail to exemplary embodiments of the invention. Wherever possible, the same reference numbers are used in the drawings and the description to refer to the same or like parts.

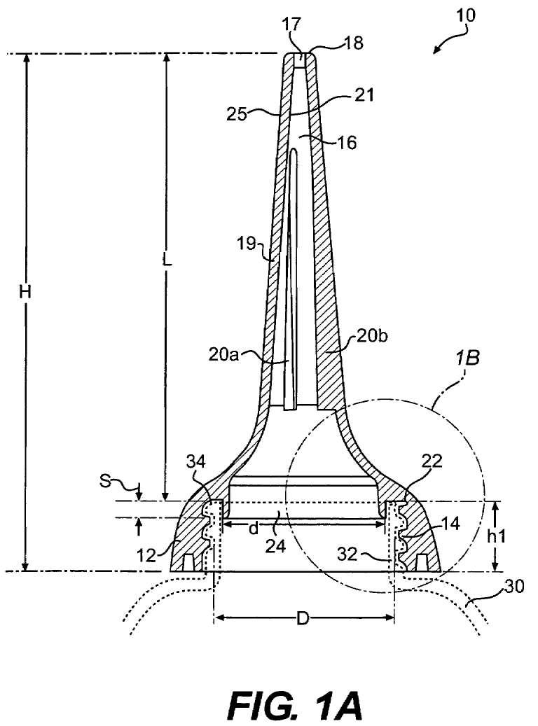

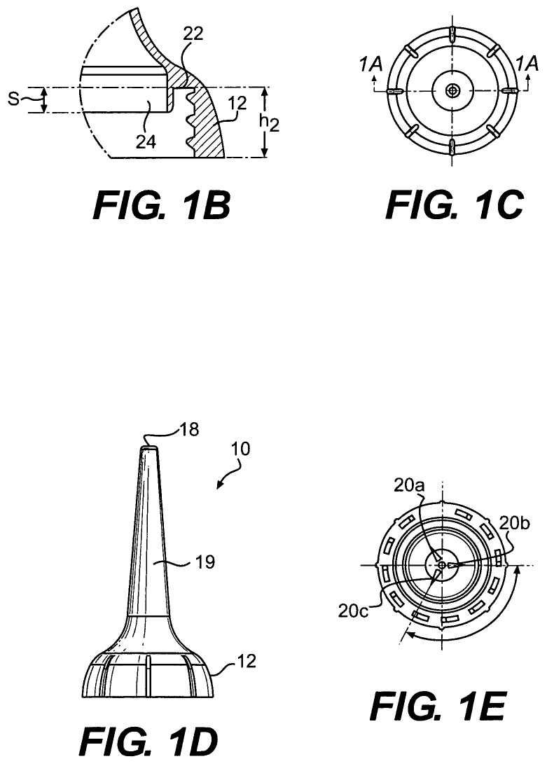

FIGS. 1A-1E show an exemplary applicator tip 10. The applicator tip 10 has a base portion 12 having a connector configured to connect the applicator tip 10 releasably to a corresponding connecting portion of a neck 32 of a container 30. As shown in FIG. 1A, the base 12 has at least one thread 14 on an inner surface that is configured to be releasably connected to at least one outer thread of a neck 32 of container 30. (FIG. 1A only shows an upper potion of the container 30 via broken lines.) Although corresponding threads are shown in the illustrated example, other forms of releasable connecting portions (e.g., interference fittings or cammed arrangements) are also possible.

The applicator tip 10 may be formed of a single, unitary piece of plastic material (e.g., a polyethylene, such as low density polyethylene). Other arrangements are also possible.

The applicator tip 10 defines a flow path 16 extending from the bottom of the applicator tip 10 (located at a bottom end of the base 12) and through an opening 17 in a top end 18 of the applicator tip 10 located opposite the base 12. Opening 17 may be sized to control (e.g., restrict and/or slow) the flow of product from tip 10. In some examples, opening 17 may be sized to control leakage and/or dripping of product from tip 10. The sizing of opening 17 may also provide product flow having various dispensing characteristics, such as smoothness of flow, coverage (e.g., dispensing pattern), accuracy, etc., in addition also possibly impacting the force used to dispense product at a given rate.

Opening 17 may have a diameter ranging from about 0.08 inches to about 0.12 inches. In one example, the diameter of opening 17 may be about 0.09 inches. In another example, the diameter of opening 17 may be about 0.095 inches. In yet another example, the diameter of opening 17 may be about 0.11 inches.

As shown in FIGS. 1A and 1E, a nozzle portion 19 of the tip 10 includes a plurality of ribs 20a, 20b, 20c extending on an inner wall surface 21 of the tip 10 toward an interior of the flow path 16. The ribs extend along a portion of a length of the flow path 16. The ribs 20a, 20b, 20c act to limit or prevent stacking of applicator tips together during tip manufacture (e.g., by limiting or preventing significant insertion of an end of another tip into the flow path 16 as tips are manufactured and placed in a common area after being formed). The ribs 20a, 20b, 20c also may act to increase the surface area of the flow path 16 and thereby restrict and/or slow the flow of product in the flow path 16. In at least some examples, the ribs 20a, 20b, 20c may enable the flow of product to be better controlled, e.g., to limit the formation of drips emerging from the opening 17 at the top end 18.

The tip may be relatively long so that roots may be reached readily easily and the configuration of the tip may enable a hair coloring product to be applied in a relatively fast and relatively drip free manner.

The applicator tip 10 has a shape and size (especially a sufficient length above the neck 32 of container 30) that facilitates using the tip 10 to form a part in hair, even when the hair is very long and thick. The shape and size (especially the length above neck 32) of the tip 10 also facilitates an individual placing opening 17 at the top end 18 of the tip 10 adjacent to the individual's scalp even when the individual has very long and thick hair. Because the shape and size of the tip 10 facilitates parting hair and/or placement of the tip adjacent to the individual's scalp, hair coloring product may be applied in a fast and efficient manner.

The shape and size (especially the length above neck 32) of the tip 10 also limits or prevents contact of the base 12 and/or the container 30 with the hair of a user. This limits or prevents product that has been applied to the hair from getting on the base 12 or the container 30, thus, limiting or preventing undesired transfer of product to, for example, skin, clothing, and/or other items that the base 12 and/or the container 30 may come in contact with during use and/or storage.

The applicator tip 10 and other associated parts of a hair coloring system may be marketed for consumer (non-professional) use, and thus, may be available for sale to consumers in stores for in-home use by individuals applying a hair coloring product to their own hair. In contrast to conventional tips supplied in home hair coloring kits intended for consumer use (which generally have a relatively short tip length due to ease of manufacture, cost, weight, packaging space limitations, etc.), the applicator tip 10 may have an appreciable length. In addition, the applicator tip 10 and the associated container 30 may have other dimensional aspects facilitating packaging and marketing of the system to consumers.

Referring to FIG. 1, the applicator tip 10 may have an overall axial height H (extending from a bottom of the base 12 to the top end 18) which is greater than about 2.3 inches (e.g., greater than about 2.5 inches or greater than about 2.7 inches). In one example, the height H may be about 2.8 inches.

As shown in FIGS. 1A and 1B, the inner threading 14 of the base 12 may extend up to an inner base end 22 that defines a surface that is placed adjacent to a top end 34 of the neck 32 of the container 30 when the container 30 and tip 10 are fully connected together (e.g., if both the tip and neck have corresponding threads, when the threads of neck 32 and tip 10 are fully engaged by normal hand tightening). The axial distance h₂ (shown in FIG. 1B) from the bottom of base 12 to the inner base end 22 may be about 0.4 inches. Other distances are also possible.

In the example shown in FIG. 1A, when container 30 and tip 10 are fully connected to one another, the top end 34 of the neck 32 either contacts the inner base end 22 or a slight gap may exist between them. In the example shown in FIG. 1A, the axial length L from the top end 18 of the tip 10 to the top end 34 of the neck 32 may be greater than about 1.9 inches (e.g., greater than about 2.1 inches or greater than about 2.3 inches). In one example, the length L may be about 2.4 inches.

In the example of FIG. 1A, the top end 34 may contact the inner base end 22 such that the axial distance h₁ from the bottom of base 12 to the top end 34 of neck 32 is substantially the same as the distance h₂. Other arrangements that lack contact between the inner base end 22 and top end 34 are also possible.

As shown in FIGS. 1A and 1B, a seal 24 may extend from the inner base end 32 toward the bottom of the tip 10 by an axial distance S that may range from about 0.08 inches to about 0.2 inches (e.g., about 0.14 inches). The seal 24 may be configured to extend into an interior of the neck 32 when the container 30 and tip 10 are fully connected to one another so as to facilitate forming a substantially fluid tight seal between an interior of container 30 and the flow path 18. The seal may have an annular shape and may have an outer diameter d of less than about 1.3 inches (e.g., less than about 1.1 inches, less than about 0.9 inches, or about 0.87 inches).

The outer diameter D of the neck 32 (as measured without taking into account the neck thread(s)) at a portion of the neck 32 including top end 34 may be less than about 1.5 inches (e.g., less than about 1.4 inches, less than about 1.3 inches, less than (or equal to) about 1.2 inches, or less than about (or equal to) about 1.1 inches).

A ratio of the length L to the outer diameter D may be greater than about 1.8, (e.g., greater than or equal to about 2.0 or greater than or equal to about 2.1). In one example, the ratio of the length L to the outer diameter D may be about 2.18.

The nozzle portion 19 also includes an outer surface 25, wherein the inner surface 21 and/or the outer surface 25 are tapered, in an axial direction, at an angle of about 8.7 degrees. Other tapers are possible.

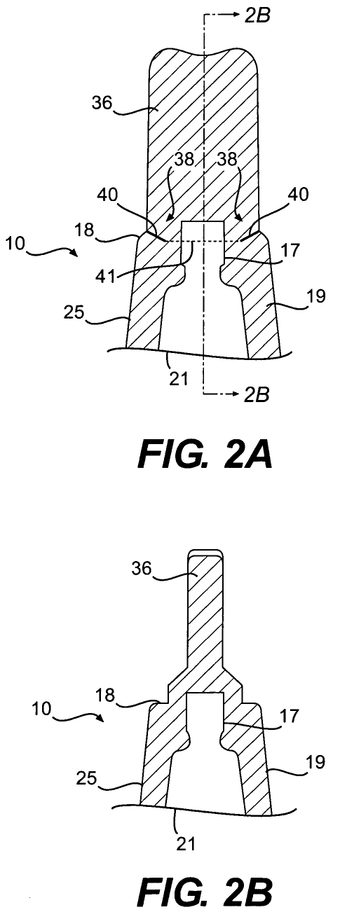

FIGS. 2A and 2B show an alternative embodiment of an applicator tip 10 configured like the tip of FIGS. 1A-1E and including a tab 36 configured to be manually removed to form the opening 17 at the top end 18 of the applicator tip 10. For example, the tab 36 may be connected to the top end 18 of the applicator tip 10 by fluidly sealed but mechanically weakened portions 38. For example, the weakened portions 38 may be weakened by scores 40, as shown in FIG. 2A. The weakened portions 38 may be configured to enable the tab 36 to be removed by twisting, folding, or otherwise bending the tab 36 relative to the nozzle portion 19 of the applicator tip 10. Upon such twisting, folding, or otherwise bending the tab 36 relative to the nozzle portion 19, the tab 36 may break off and cause the top end of the tip 10 to be formed approximately along an area designated by a dashed line 41 in FIG. 2A.

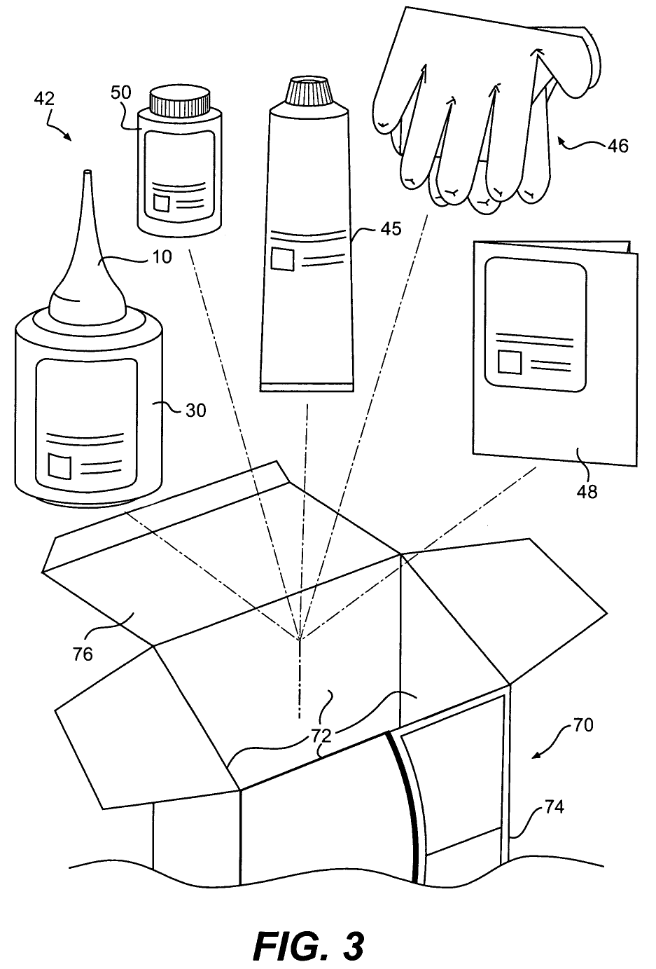

FIG. 3 depicts an embodiment of a hair coloring system 42. The system 42 includes at least one component of a hair coloring product, such as a hair dye and/or a hair lightener (e.g., contained in container 30). The system 42 also includes the container 30, the applicator tip 10, and, in some examples, other items described in greater detail below.

In certain examples, the system 42 may be packaged and, thus, may include a packaging 70. The packaging 70 includes one or more walls 72 defining a packaging interior partially shown in FIG. 3. The walls 72 may be formed of a variety of different materials, such as carton board, cardboard, paper, plastic, and/or any other material. In the exemplary embodiment of FIG. 3, the packaging 70 may be in the form of a box 74 including a top flap 76 movable between an open position (shown in FIG. 3) permitting access to the interior via an opening of the packaging, and a closed position (not shown) closing the opening leading to the interior. Several alternative packaging arrangements other than boxes and/or boxes having an opening and closable flap are also possible. For example, the packaging could be in any form, including a plastic “blister” package.

As shown in the example of FIG. 3, the parts of the hair coloring system 42 contained in the packaging 70 may include one or more items, such as containers 30, 50, the applicator tip 10, a tube 45, a pair of gloves 46, and an insert 48 including instructions for using the hair coloring system to treat hair. The containers 30 and 50 may contain components of a hair coloring product. For example, one of the containers (e.g., container 30) may contain a color component and the other container (e.g., container 50) may contain a developer component. The color component and the developer component may be mixed to create a hair dye, which may be dispensed from container 30 using applicator tip 10. In some examples, the hair dye formed by mixing the contents of the containers 30 and 50 may be in a creme form (e.g., having a viscosity that limits dripping). Alternatively, the component(s) of the hair coloring product of the system may be a pre-mixed and/or pre-formulated dye and/or hair lightener. The tube 45 may contain a hair conditioner.

The container 30 may be collapsible by applying manual pressure so as to permit product dispensing through tip 10. For example, container 30 may be a squeeze bottle.

Although containers 30 and 50 and tube 45 have been shown as having a particular size, shape and style, and have been described as containing particular components and/or products, containers 30 and 50 and tube 45 may have any suitable size, shape, and style and may contain any of the components and/or products disclosed herein or other components/products. Further, in some embodiments, one or more of containers 30 and 50 and tube 45 may be packaged empty, and thus, may be available to be at least partially filled with one or more components/products during use. For example, two or more hair coloring components may be added to container 30 at the time of use (or even before the system parts are placed in the packaging 70). The hair coloring components may be individually added to container 30 or mixed separately and added to container 30 in a mixed form.

The system 42 may also include many additional or alternative items and/or forms of products. For example, one or more additional applicators, such as a brush and/or comb (not shown), may be provided.

The system 42 may include one or more gloves, such as the pair of gloves 46 shown in FIG. 3. The pair of gloves 46 may be any type of protective and, in some embodiments, disposable gloves. For example, in one embodiment, the pair of gloves 46 may be surgical-type latex gloves. In at least some examples, the pair of gloves 46 could be made from any material suitable for preventing hair product from getting on a user's hands, including various types of rubber, plastic, cloth, or other materials.

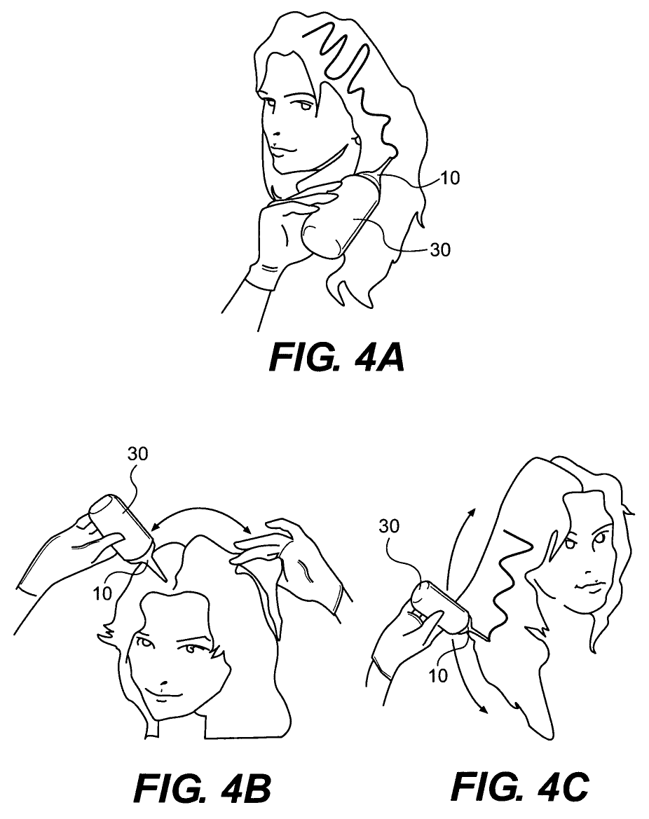

FIGS. 4A, 4B, and 4C show exemplary uses of the system 42. In an exemplary method of coloring hair performed by an individual coloring the individual's own hair, the applicator tip 10 may be used to dispense hair coloring product from the container 30. For example, as shown in FIG. 4A, the system 42 may be used to evenly color all of a user's hair (e.g., when the user has never colored their hair before or if they have not colored their hair for more than 3 months). To evenly color all of a user's hair, the system 42 may be used to saturate all of the user's hair. The hair coloring product may be left in for a period of time (e.g., 30 minutes) to take effect.

Alternatively, as shown in FIGS. 4B and 4C, the system 42 may be used to apply a “touch-up” application of a hair coloring product to a user's hair (e.g., when hair grows out after a previous coloring involving the same shade of hair coloring product being applied in the touch-up). Touching-up a user's hair may include placing the opening 17 of the tip 10 adjacent to the individual's own scalp, as shown in FIG. 4B so as to apply the hair coloring to the individual's hair. In order to place the opening 17 adjacent to the individual's own scalp (and/or to better define an area where product is to be applied), the user may use the tip 10 to form a part in their hair, as shown in FIG. 4B. The hair coloring product may be left in for a period of time (e.g., 20 minutes). Touching-up may also include second step, as shown in FIG. 4C. In the second step, the user may saturate the rest of their hair with the hair coloring product and leave it in for a period of time (e.g., 10 minutes). The user may then complete the hair coloring procedure by rinsing the hair coloring product out of their hair and conditioning their hair with, for example, a conditioner that may be provided in the system 42.

It will be apparent to those skilled in the art that various modifications and variations can be made to the structures and methodologies described herein. Thus, it should be understood that the invention is not limited to the subject matter discussed in the specification. Rather, the present invention is intended to cover modifications and variations.

What is claimed is:

1. A hair coloring system, comprising:
at least one component of a hair coloring product;
a container configured to contain the hair coloring product, the container comprising
a neck having a connecting portion and a top neck end; and
an applicator tip for applying the hair coloring product to hair, the applicator tip comprising
a base portion configured to connect the applicator tip releasably to the connecting portion of the neck of the container, and
a nozzle portion extending from the base portion to a top end of the tip,
wherein the applicator tip and the container are configured such that when the applicator tip is fully connected to the connecting portion of the neck of the container, an axial length from the top neck end to the top end of the tip is greater than about 1.9 inches, and
wherein the applicator tip and the container are configured such that a ratio of said axial length to an outer diameter of a portion of the neck including the top neck end is greater than about 1.8.

2. The system of claim 1, wherein said axial length is greater than about 2.3 inches and wherein said ratio is greater than or equal to about 2.1.

3. The system of claim 2, wherein the tip has an overall axial height greater than about 2.7 inches.

4. The system of claim 1, wherein the applicator tip further comprises a seal configured to extend into the neck of the container when the applicator tip is fully connected to the connecting portion, and wherein the seal has an outer diameter of less than about 1.3 inches.

5. The system of claim 1, wherein the applicator tip comprises an opening at the top end of the tip.

6. The system of claim 1, wherein the applicator tip comprises a tab configured to be manually removed to form an opening at the top end of the applicator tip.

7. The system of claim 1, wherein an inner surface of the applicator tip defines a flow path, and wherein the nozzle portion comprises axially-oriented ribs extending along the inner surface.

8. The system of claim 1, wherein the nozzle portion includes an inner surface and an outer surface, and wherein at least one of the inner surface and the outer surface is tapered, in an axial direction, at an angle of about 8.7 degrees.

9. The system of claim 1, wherein the hair coloring product is in the form of a creme.

10. The system of claim 1, further comprising:
a pair of gloves;
at least one additional component of the hair coloring product;
instructions for using the hair coloring system to color hair; and
a packaging,
wherein the components of the hair coloring product, the container,
the applicator tip, and the pair of gloves are contained in the packaging.

11. A method of coloring hair performed by an individual, comprising:
dispensing hair coloring product from an opening at the top end of the applicator tip of the system of claim 1; and
placing the opening adjacent to the individual's scalp so as to apply the hair coloring product to the individual's hair.

12. The method of claim 11, further comprising using the applicator tip to form a part in the individual's hair.

13. A hair coloring system, comprising:
first and second components, wherein the first and second components form a hair coloring product by mixing the first and second components together;
a container configured to contain the hair coloring product, the container comprising
a neck having a connecting portion and a top neck end,
wherein an outer diameter of a portion of the neck including the top neck end is less than about 1.5 inches; and
an applicator tip for applying the hair coloring product to hair, the applicator tip comprising
a base portion configured to connect the applicator tip releasably to the connecting portion of the neck of the container, and
a nozzle portion extending from the base portion to a top end of the tip; and
a packaging,
wherein the first and second components of the hair coloring product, the container, and the applicator tip are contained in the packaging, and
wherein the applicator tip and the container are configured such that when the applicator tip is fully connected to the connecting portion of the neck of the container, an axial length from the top neck end to the top end of the tip is greater than about 1.9 inches.

14. The system of claim 13, wherein said diameter is less than or equal to about 1.2 inches.

15. The system of claim 14, wherein said axial length is greater than about 2.3 inches.

16. The system of claim 13, wherein the applicator tip further comprises a seal configured to extend into the neck of the container when the applicator tip is fully connected to the connecting portion, and wherein the seal has an outer diameter of less than about 1.3 inches.

17. The system of claim 1, wherein the applicator tip comprises an opening at the top end of the tip.

18. The system of claim 1, wherein an inner surface of the applicator tip defines a flow path, and wherein the nozzle portion comprises axially-oriented ribs extending along the inner surface.

19. The system of claim 1, further comprising a pair of gloves contained in the packaging.

20. A method of coloring hair performed by an individual, comprising:
dispensing hair coloring product from an opening at the top end of the applicator tip of the system of claim 1; and
placing the opening adjacent to the individual's scalp so as to apply the hair coloring product to the individual's hair.
